(19) United States
(12) Patent Application Publication
(10) Pub. No.: US 2023/0050817 A1
(43) Pub. Date: Feb. 16, 2023

(54) METHOD FOR PREPARING AN AUDIOGRAM OF A TEST SUBJECT BY USE OF A HEARING INSTRUMENT

(71) Applicant: Sivantos Pte. Ltd., Singapore (SG)

(72) Inventor: Umut Gökay, Mainz (DE)

(21) Appl. No.: 17/884,744

(22) Filed: Aug. 10, 2022

(30) Foreign Application Priority Data
Aug. 10, 2021 (DE) 10 2021 208 735.3

(51) Int. Cl. A61B 5/12; A61B 5/00

(52) U.S. Cl. CPC A61B 5/123; A61B 5/7217; A61B 5/6803; A61B 5/6817; A61B 2562/0204

(57) ABSTRACT

A method for preparing an audiogram of a test subject by use of a hearing instrument. A sound signal is recorded in an auditory canal of the test subject at least partially closed by the hearing instrument by a first electroacoustic input transducer of the hearing instrument. A first input signal is generated therefrom. A test sound is generated by an electroacoustic output transducer of the hearing instrument and output into the auditory canal of the test subject at least partially closed by the hearing instrument. A hearing threshold of the test subject at at least one test frequency is ascertained on a basis of a reaction of the test subject to the test sound and by use of the first input signal.

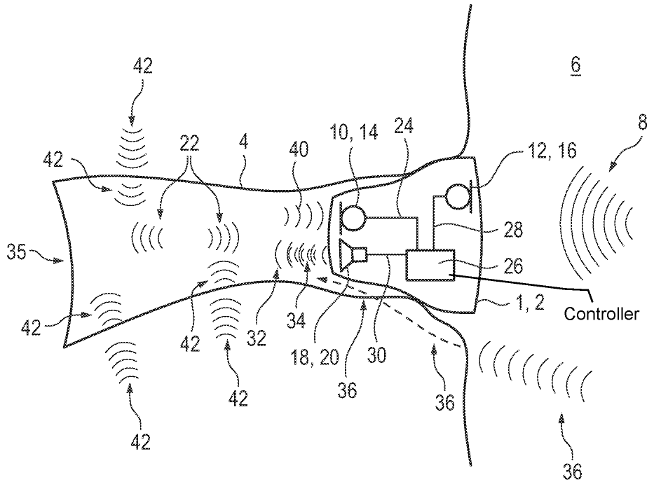

### CROSS-REFERENCE TO RELATED APPLICATION

This application claims the priority, under 35 U.S.C. § 119, of German Patent Application DE 10 2021 208 735.3, filed Aug. 10, 2021; the prior application is herewith incorporated by reference in its entirety.

### FIELD AND BACKGROUND OF THE INVENTION

The invention relates to a method for preparing an audiogram of a test subject by means of a hearing instrument. A test sound is generated by an electroacoustic output transducer of the hearing instrument and is output into an auditory canal of the test subject at least partially closed by the hearing instrument, and a hearing threshold of the test subject at at least one test frequency is ascertained on the basis of a reaction of the test subject to the test sound. The invention furthermore relates to a hearing instrument, by means of which a hearing threshold of a test subject at at least one test frequency may be ascertained.

A hearing instrument in general contains any device which is provided and configured to generate a corresponding output sound from an electrical output signal by means of an electroacoustic output transducer and to supply it to a sense of hearing of a user. In particular a loudspeaker can be used here as such an output transducer, however, in particular thermoacoustic transducers or bone vibrators can also be used. A hearing instrument can only be configured for generating the output sound on the basis of audio data, on the one hand, thus in the form of a wireless headphone, in particular in the form of an earbud headphone, for example. In this case, an output sound is generated on the basis of audio data which can be provided, for example, by music and which were previously stored, or are also transmitted via a corresponding antenna to the hearing instrument (via stream).

A hearing instrument can also be provided as a hearing aid, however, which is configured to correct or at least partially compensate for a hearing impairment of a user, in that, for example, an ambient sound is converted into a corresponding electrical input signal by means of at least one electroacoustic input transducer, which is processed in the hearing aid according to the audiological requirements of the user and in particular is amplified by frequency band, so that the processed input signal is supplied to the sense of hearing of the user as output sound via the electroacoustic output transducer.

In particular for the correction of a hearing impairment of the user in the case of a hearing aid (in the “narrower meaning”), an audiogram of the user is usually prepared. This indicates the respective hearing threshold of the user at the relevant frequency for individual test frequencies, which preferably cover the entire acoustic spectrum of human hearing sensation, so that the hearing capability of the user can in particular be compared to the hearing capability of persons having normal hearing, to obtain therefrom conclusions for the above-mentioned individualized signal processing in the hearing aid. However, the preparation of an audiogram can also be advantageous for an adaptation of a hearing instrument (in the “broader meaning”) to a user to improve the perception of sound, thus, for example, for better acoustic performance in specific frequency ranges.

The measurement of the hearing threshold at the individual test frequencies is usually carried out for this purpose at an audiologist or established acoustician, wherein often a test probe having a sound generator is inserted into an auditory canal of the user. Individual test tones at the respective test frequencies are each generated with continuously increasing or decreasing sound level, so that the user can indicate the recognition from when he hears or no longer hears the test tone at a test frequency. One disadvantage here is that such measurements of the audiogram are to be carried out at the or by the audiologist/acoustician.

### SUMMARY OF THE INVENTION

The invention is therefore based on the object of specifying a method by which an audiogram of a wearer of the hearing instrument may be prepared by means of a hearing instrument in the simplest possible and nonetheless reliable manner and independently of the situation as much as possible. The invention is furthermore based on the object of specifying a hearing instrument which is configured for the simplest and most reliable possible preparation of an audiogram of a wearer.

The first-mentioned object is achieved according to the invention by a method for preparing an audiogram of a test subject by means of a hearing instrument. A sound signal is recorded in an auditory canal of the test subject at least partially closed by the hearing instrument by a first electroacoustic input transducer of the hearing instrument. A first input signal is generated therefrom, wherein a test sound is generated by an electroacoustic output transducer of the hearing instrument and is output into the auditory canal of the test subject at least partially closed by the hearing instrument. A hearing threshold of the test subject at at least one test frequency is ascertained on the basis of a reaction of the test subject to the test sound and by means of the first input signal. Advantageous embodiments, and inventive embodiments which are partially considered per se, are the subject matter of the dependent claims and the following description.

The second-mentioned object is achieved according to the invention by a hearing instrument, containing a first electroacoustic input transducer, which is configured, when the hearing instrument is worn as intended by a test subject, to record a sound signal in an auditory canal of the test subject, and to generate a first input signal therefrom. The hearing instrument further having an electroacoustic output transducer, which is configured to generate a test sound and, when the hearing instrument is worn as intended, to output it into the auditory canal of the test subject. A control device is provided, which is configured to register a reaction of the test subject to the test sound and to ascertain a hearing threshold of the test subject at at least one test frequency on the basis of the reaction of the test subject to the test sound and by means of the first input signal.

The hearing system according to the invention shares the advantages of the method according to the invention. The advantages indicated for the method and for its refinements can be transferred accordingly to the hearing system.

The control device preferably contains at least one signal processor and a working memory addressable by the signal processor, which are configured to carry out the mentioned signal processing steps. The hearing system can be provided, on the one hand, by the hearing instrument, or can also have an auxiliary device connectable for data processing to the hearing instrument, for example, a smart phone or a tablet PC, wherein the control device can be provided in the latter case by a control unit in the hearing instrument and/or a control unit on the auxiliary device.

An electroacoustic input transducer contains in this case in particular any transducer which is configured to generate an electrical audio signal from an ambient sound, so that air movements and air pressure variations induced by the ambient sound at the location of the transducer are reproduced by corresponding oscillations of an electrical variable, in particular a voltage in the generated audio signal. In particular, the electroacoustic input transducer can be provided by a microphone. Accordingly, an electroacoustic output transducer contains any transducer which is configured to generate an output sound from an electrical output signal, thus in particular a loudspeaker (such as a Balanced Metal Case Receiver), but also, for example, a thermoacoustic transducer.

Recording of a sound signal in an auditory canal of the test subject at least partially closed by the hearing instrument by the first electroacoustic input transducer of the hearing instrument in particular contains that the hearing instrument is worn by the test subject, in particular in a way corresponding to the intended use of the hearing instrument, on an ear and in this case the auditory canal of the relevant ear is at least partially closed to the outside at least by a part of the hearing instrument. In particular, a part of the hearing instrument can be fixedly applied in the concha by pressure and/or a part can protrude into the auditory canal and can be fixedly applied there by pressure.

The hearing instrument is preferably configured here so that the first electroacoustic input transducer, when the hearing instrument is worn as intended on the ear, is oriented into the relevant auditory canal. The sound signal recorded by the first electroacoustic input transducer is in particular provided by that sound which propagates between the eardrum and the hearing instrument, and can contain a high proportion of structure-borne sound, which cannot escape into the surroundings due to the partial closure of the auditory canal by the hearing instrument (so-called occlusion effect). The first input signal therefore in particular contains signal contributions of the structure-borne sound.

The test sound preferably has a defined first signal contribution at the at least one test frequency, thus in particular a defined sound level in the range of the first test frequency. In particular, this defined first signal contribution or defined sound level in the range of the first test frequency can be varied, thus, for example, can be increased continuously from a sound level which is in all probability inaudible to every test subject, or can be continuously increased from a sound level which is in all probability audible to every test subject. The hearing instrument is preferably configured here so that the electroacoustic output transducer is oriented into the relevant auditory canal when the hearing instrument is worn as intended on the ear.

The reaction of the test subject to the test sound can be detected here in particular as a speech signal by a second electroacoustic input transducer of the hearing instrument, which is oriented into the free surroundings of the hearing instrument and is configured to generate a second input signal from the ambient sound of the hearing instrument. The second input signal can then be analyzed accordingly by a control device of the hearing instrument for speech commands, which include a reaction to the test sound. The reaction can in particular also be detected by means of an auxiliary device, however, which is activated accordingly by the test subject, for example, by means of a corresponding app for detecting the reaction via a display screen input of the test subject. The reaction can also be detected, however, as a movement of the test subject by means of a correspondingly configured sensor of the hearing instrument, thus, for example, as a nod of the head or shake of the head to signal that the test sound is heard or is not (is no longer) heard, for example, by means of a movement and/or acceleration sensor and/or a gyroscope.

The hearing threshold at the at least one test frequency can be ascertained in particular in that in the above-described manner the test sound is output with variable, preferably continuously increasing or decreasing sound level in the range of the first test frequency into the auditory canal of the test subject, and the test subject reveals via the reaction from when the test sound becomes audible (increasing) or when the test sound is no longer audible (decreasing).

The first input signal, on the one hand, can be used here for the determination of the hearing threshold in such a way that a type of “noise background” in the auditory canal is ascertained on the basis of the first input signal before an emission of the test sound, and the noise background is also taken into consideration for the determination of the sound level at the at least one test frequency (the other significant signal contribution at the test frequency originates from the test sound, the defined sound level of which is to be offset by the noise background for the final sound level at the test frequency). On the other hand, on the basis of the first input signal, the defined first signal contribution of the test frequency in the test sound can also be adjusted adaptively and in particular during the output of the test sound, for which purpose in particular filtering of the first input signal with respect to the first signal contribution can take place at the at least one test frequency. In addition, it is possible to carry out an active occlusion suppression on the basis of the first input signal. For this purpose, a correction signal for the occlusion suppression is generated on the basis of the first input signal, which is converted by the electroacoustic output transducer into a correction sound. The correction signal can be superimposed here on the signal provided for the generation of the test sound for the output.

The audiogram is determined for the at least one test frequency on the basis of and preferably directly by the hearing threshold thus ascertained. The audiogram can now be completely prepared in the described manner in that a plurality of test frequencies is specified, and successively a corresponding test sound having a defined signal contribution at the respective test frequency is generated and output into the auditory canal of the test subject, and for each of the test frequencies the hearing threshold is ascertained on the basis of the respective reaction of the test subject to the relevant test sound and on the basis of the first input signal. The test frequencies preferably completely cover the human hearing spectrum here, and moreover enable the most advantageous possible frequency resolution of the hearing spectrum (thus, for example, an essentially uniform number of test frequencies for any arbitrarily selected octave).

The hearing threshold ascertained as described for the at least one test frequency can also be used, however, to replace the corresponding data in an already existing or predefined audiogram, or as a supplement to such an audiogram.

In particular for a hearing instrument, which is to be worn on an ear for the intended operation so that the relevant auditory canal is closed to a significant extent, a measurement of a hearing threshold by means of the hearing instrument can be obstructed or also corrupted by the closure of the auditory canal as a result of occlusion effects. The proposed method solves this problem in that noises in the auditory canal, which do not belong to the test sound and in particular to the first signal contribution of the test sound at the at least one test frequency, can be actively attenuated, or their contribution to the overall sound level in the auditory canal in the range of the at least one test frequency can be taken into consideration. The hearing threshold may also be ascertained by the hearing instrument in this way as a result of the avoidance of the above-mentioned corruptions. The test subject can thus be spared having to find an audiologist or an established acoustician for a recreation of an audiogram or also only an adjustment or correction of the audiogram.

The test sound preferably has a defined first signal contribution at the at least one test frequency, wherein a second signal contribution of the sound signal in the auditory canal at the at least one test frequency is ascertained on the basis of the first input signal, and wherein at least the second signal contribution is taken into consideration for an ascertainment of the hearing threshold in the at least one test subject. This can take place in particular in that the second signal contribution detects the overall sound in the auditory canal at the at least one test frequency, which is composed of the defined first signal contribution of the test sound, and a sound contribution which is substantially caused by structure-borne sound and possibly also contains ambient sound, which enters the auditory canal past the usually incomplete closure of the auditory canal by the hearing instrument. A priori, only the first signal contribution in the test sound generated by itself is known in the hearing instrument, due to which a reaction to the sum of the test sound and the other mentioned contributions can possibly result in an incorrect, in particular excessively low hearing threshold. This can be corrected by the consideration of the second signal contribution, in that the first signal contribution is adjusted accordingly.

The second signal contribution can however—in particular after filtering of the first input signal with respect to the first signal contribution—also only have the contributions caused by structure-borne sound and possibly ambient sound, so that in this case the first and the second signal contribution are to be used for the determination of the sound level in the auditory canal (and accordingly for the correct hearing threshold).

A correction signal for an active occlusion suppression is advantageously generated on the basis of the first input signal. A correction sound is generated by the electroacoustic output transducer of the hearing instrument on the basis of the correction signal to compensate for a structure-borne sound in the sound signal of the auditory canal and is output into the auditory canal (at least partially closed by the hearing instrument) of the test subject. An active occlusion suppression ascertains noises in a substantially closed auditory canal, which are based on structure-borne sound, and then generates a compensation sound in the auditory canal which compensates as much as possible for the structure-borne sound. In the present case, the signal contributions based on structure-borne sound in the auditory canal can be ascertained on the basis of the first input signal, which is preferably to be filtered for this purpose by the defined first signal contribution caused by the test sound by means of corresponding filtering. For the residual signal contributions thus ascertained, a corresponding, preferably counter-phase correction signal is generated for compensation, which the electroacoustic output transducer of the hearing instrument converts into a correction sound that is output into the auditory canal.

The compensation thus described of structure-borne sound as a result of occlusion effects, which preferably takes place in a broadband manner, reduces the sound level in the auditory canal, due to which, on the one hand, better concentration on the test sound and in particular its first signal contribution at the test frequency is enabled for the test subject, and, on the other hand, masking effects due to sound events beyond the test frequency can be prevented, which become relevant in particular at low sound levels of the test sound (as nearly necessarily occur to ascertain the hearing threshold). A possible additionally occurring compensation of noises caused by ambient sound can have a further positive effect on the ascertainment of the hearing threshold here.

The correction sound is advantageously generated and output here as a broadband sound signal, wherein a residual noise signal not filtered by the active occlusion suppression is ascertained as the second signal contribution on the basis of the first input signal. This second signal contribution is taken into consideration in particular for the generation of the test sound, in that the first signal contribution of the test sound at the test frequency is set in accordance with the second signal contribution, to achieve the desired sound level in the auditory canal in the range of the test frequency.

An ambient sound of the hearing instrument is advantageously recorded by a second electroacoustic input transducer of the hearing instrument, and a second input signal is generated therefrom, wherein an active noise suppression (“active noise canceling”, ANC) is carried out on the basis of the second input signal by the electroacoustic output transducer of the hearing instrument in addition to the output of the test sound. An ANC ascertains noises in the ambient sound, and then generates a compensation sound in the auditory canal, which compensates as much as possible for the noise entering the auditory canal from the surroundings. In the case that the hearing instrument is provided as a hearing aid, it already has at least one electroacoustic input transducer for recording ambient sound for normal operation. However, hearing instruments without specific correction of a hearing impairment often also have such an input transducer for recording ambient sound, for example, to be able to receive speech commands of the user, or also for telephony functions. The correction signal generated for the ANC can be superimposed on the signal provided for the generation of the test sound for output. Due to the ANC, the test subject is subjected to the ambient noises to a lesser extent during the ascertainment of the hearing threshold, so that corruptions and also masking effects are also further suppressed here.

The active noise suppression is expediently concentrated here on a frequency range around the at least one test frequency. An ANC typically has a specific operating range, in which a noise is suppressed particularly efficiently. A narrowband frequency range of, for example, 50 Hz or 100 Hz or 250 Hz around the test frequency may be optimized better for a complete noise suppression here, while a more broadband ANC, for example, overall achieves a stronger reduction of the total noise energy, perhaps, wherein possibly a greater residual noise can remain particularly in the surroundings of the test frequency, which is not eliminated by the ANC. The bandwidth of the ANC is therefore reduced here for the purpose of the most complete possible noise suppression at the test frequency and preferably in its immediate surroundings. An ANC supplies the best results with strongly predictable signals, which have a high tonal component and therefore a clear localization in the frequency spectrum. For the ascertainment of the hearing threshold this means that the ANC is preferably to be applied when a strongly tonal noise signal occurs in the range of the test frequency in the surroundings of the test subject.

It has proven to be advantageous here if the active noise suppression is concentrated on a frequency range around the at least one test frequency, and the active occlusion suppression by the correction signal takes place in a broadband manner. The compensation signal of the active occlusion suppression, in contrast to the case of the ANC, is preferably generated in a feedback loop, due to which other possibilities result in the compensation of the noises. Since occlusion effects often occur with greater spectral width, it can be advantageous not to restrict the compensation by means of the correction sound to specific frequency ranges. In particular, the correction signals of the ANC and the active occlusion suppression can be superimposed here for the output on the signal provided for the generation of the test sound.

In one advantageous embodiment, the hearing instrument is configured as a hearing aid, and furthermore contains a second electroacoustic input transducer, which is configured to record an ambient sound of the hearing aid, and to generate a second input signal therefrom. The control device is furthermore configured to generate an output signal on the basis of the second input signal and to supply it to the electroacoustic output transducer for conversion into an output sound signal. In particular for hearing aids, the creation of an audiogram by a user and without audiological supervision is advantageous.

The control device is expediently furthermore configured to carry out active noise suppression on the basis of the second input signal by means of the electroacoustic output transducer in addition to the output of the test sound.

Other features which are considered as characteristic for the invention are set forth in the appended claims.

Although the invention is illustrated and described herein as embodied in a method for preparing an audiogram of a test subject by means of a hearing instrument, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

The FIGURE of the drawing is a sectional view of a hearing instrument, which closes an auditory canal, and by means of which a hearing threshold of a user can be ascertained according to the invention.

### DETAILED DESCRIPTION OF THE INVENTION

Referring now to the FIGURE of the drawing in detail 1 thereof, there is shown schematically and not true to scale, a sectional illustration of a hearing instrument 1, which is configured in the present case as a hearing aid 2. The hearing aid 2 is inserted into an auditory canal 4 and is seated fixedly in its position due to pressure on the skin at the auditory canal 4. The auditory canal 4 is substantially closed in relation to the surroundings 6 in this way, so that even a low proportion of an ambient sound 8 enters the auditory canal 4 past the hearing aid 2—via an incomplete closure of the auditory canal 4 as a result of a seat of the hearing aid 2 not applied exactly in the auditory canal 4—and through the thin skin parts at the entry of the auditory canal 4.

The hearing aid 2 has a first electroacoustic input transducer 10 and a second electroacoustic input transducer 12, which are provided in the present case as a first and second microphone 14 and 16, and an electroacoustic output transducer 18, which is provided as a loudspeaker 20. The first microphone 14 and the loudspeaker 20 are directed into the auditory canal 4 when the hearing aid 2 is worn as intended and the second microphone 16 is directed toward the free surroundings 6 of the hearing aid 2.

The first microphone is configured to record a sound signal 22 in the auditory canal 4 and to generate a first input signal 24 therefrom, which is supplied to a control device 26. The second microphone 16 is configured to generate a second input signal 28 from the ambient sound 8, which is also supplied to the control device 28. In particular, the hearing aid 2 can also have a further electroacoustic input transducer (not shown) oriented toward the free surroundings 6, which is configured to generate a further input signal from the ambient sound 8.

In normal operation of the hearing aid 2, the second input signal (and possibly said further input signal) is processed specifically by frequency band in the control device 26 and in particular amplified, and an output signal 30 is generated therefrom, which is supplied to the loudspeaker 20. For this purpose, the control device 26 can in particular have a signal processor (not shown) and a working memory addressable by the signal processor. The loudspeaker 20 generates an output sound signal (not shown) from the output signal 30. A hearing impairment of a user of the hearing aid 2 is taken into consideration here for the generation of the output signal 30 by the control device 26 in that, inter alia, a frequency band-specific amplification takes place according to the audiological requirements of the user to compensate for his hearing impairment. For this purpose, an audiogram is required in the hearing aid 2 as the information of these audiological requirements.

Such an audiogram, which indicates the hearing threshold of the user for individual frequencies, is typically prepared at an audiologist or an acoustician, which the user seeks out for this purpose.

However, the hearing aid 2 is configured by several special features described hereinafter so that an audiogram can also be prepared with the aid of the hearing aid 2, or an already existing audiogram (and an audiogram preferably stored in a nonvolatile memory of the hearing aid 2, in particular also for use in running operation) can be updated for some frequencies.

To be able to ascertain a hearing threshold of the user (and thus to be able to use it for an audiogram) by means of the hearing aid 2, the hearing aid 2 is advantageously designed to suppress interfering noises, which could make a measurement of a hearing threshold more difficult (“inconclusive result”) or could also corrupt it (incorrect result), and which typically do not occur to a noteworthy extent in the surroundings specifically configured for such measurements at an audiologist or acoustician.

A test sound 32 is output by the electroacoustic output transducer 18, which is generated by corresponding contributions in the output signal 30 converted by the electroacoustic output transducer 18. The test sound 32 has a defined first signal contribution 34 in the range of a test frequency here, for which the hearing threshold is to be ascertained. The test sound 32 having the first signal contribution 34 propagates here through the auditory canal 4 to the eardrum 36 of the user and is perceived by the user depending on the sound level of the first signal contribution 34. For a measurement of the hearing threshold at the test frequency, the first signal contribution 34 is varied here (thus, for example, continuously increased from a non-perceptible starting value, or continuously decreased from a well perceptible starting value), so that the user can convey a change of his perception (test sound becomes audible or becomes inaudible).

This can take place, for example, by means of a corresponding speech input via the second electroacoustic input transducer 12, wherein the second input signal 28 is to be analyzed during the output of the test sound 32 for such a speech input. It is also possible to carry out a user input by means of a smart phone, a tablet PC (both not shown), or the like, which is preferably wirelessly connected to the hearing aid 2, wherein in particular a correspondingly configured app can be used for the input and also for the selection of test frequencies or the like.

In order that the measurement of the hearing threshold is not made more difficult or corrupted by external interference noises 36 in the ambient sound 8, which propagate via the schematically shown sound path 38 in the auditory canal 4 (and thus further to the eardrum 35 of the user), the external interference noises 36 are compensated via an ANC, in that, on the basis of the second input signal 28 generated by the second electroacoustic input transducer 12 (which does detect the ambient sound 8 and thus the external interference noises 36), a compensation signal (not shown) is generated in the control device 26, which is incorporated into the output signal 30. The compensation signal is generated in phase and amplitude here in such a way that the associated signal contributions in the output sound signal generated by the loudspeaker 20 compensate as completely as possible for the components of the external interference noises 36 which are propagated via the sound path 38 in the auditory canal 4.

Furthermore, a second signal contribution 40 in the sound signal 22 in the auditory canal 4 is ascertained by means of the first electroacoustic input transducer 10 at the test frequency of the test sound 32. This is carried out with the goal of suppressing a structure-borne sound 42 penetrating into the auditory canal 4 by way of an active occlusion suppression. The component of structure-borne sound 4 in the sound signal 22 can be detected by the first input signal 24 generated by the first electroacoustic input transducer 10, so that, in dependence on the detected structure-borne sound 42, a correction signal (not shown) can be generated, which is incorporated in the output signal 30. The output sound signal correspondingly generated by the electroacoustic output transducer 18 then contains a correction sound, which compensates for the structure-borne sound 42 as completely as possible. A detection of the structure-borne sound 42 can preferably take place chronologically separated from an output of the test sound 32, so that, for example, shortly before such an output, the structure-borne sound 42 to be compensated is ascertained, and a corresponding compensation takes place by means of the correction sound during the output of the test sound. The output sound signal generated by the electroacoustic output transducer 18 can thus detect the test sound 32 and contributions of the active occlusion suppression to compensate for the structure-borne sound 42 in the sound signal 22 in the auditory canal 4 and contributions of an ANC to compensate for the external interference noises 36 in the ambient sound 8.

Alternatively thereto, the structure-borne sound 42 can also take place (not shown) during the output of the test sound 32. In this case, the second signal contribution 40 of the sound signal 22 in the auditory canal 4 also contains the first signal contribution 34 of the test sound 32 at the test frequency. The second signal contribution 40 can then be used to set the first signal contribution 34 in the test sound 32 so that a desired sound level occurs in each case at the eardrum 35 in the range of the test frequency as the sum signal made up of structure-borne sound 42 and first signal contribution 32. The second signal contribution 34 is thus used here for a direct correction of the first signal contribution 32 in the test sound 32.

Although the invention was illustrated and described in more detail by the preferred exemplary embodiment, the invention is not thus restricted by the disclosed examples and other variations can be derived therefrom by a person skilled in the art without leaving the scope of protection of the invention.

The following is a summary list of reference numerals and the corresponding structure used in the above description of the invention:
1 hearing instrument
2 hearing aid
4 auditory canal
6 free surroundings
8 ambient sound
10 first electroacoustic input transducer
12 second electroacoustic input transducer
14 first microphone
16 second microphone
18 electroacoustic output transducer
20 loudspeaker
22 sound signal
24 first input signal
26 control device
28 second input signal
30 output signal
32 test sound
34 first signal contribution
35 eardrum
36 external interference noises
38 sound path
40 second signal contribution
42 structure-borne sound

1. A method for preparing an audiogram of a test subject by means of a hearing instrument, which comprises the steps of:
recording a sound signal in an auditory canal of the test subject at least partially closed by the hearing instrument by a first electroacoustic input transducer of the hearing instrument;
generating a first input signal from the sound signal;
generating a test sound by an electroacoustic output transducer of the hearing instrument;
outputting the test sound into the auditory canal of the test subject at least partially closed by the hearing instrument; and
ascertaining a hearing threshold of the test subject at at least one test frequency on a basis of a reaction of the test subject to the test sound and by means of the first input signal.

2. The method according to claim 1, wherein:
the test sound has a defined first signal contribution at the at least one test frequency;
a second signal contribution of the sound signal in the auditory canal at the at least one test frequency is ascertained on a basis of the first input signal; and
at least the second signal contribution is taken into consideration for an ascertainment of the hearing threshold in the at least one test subject.

3. The method according to claim 2, which further comprises:
generating a correction signal for an active occlusion suppression on a basis of the first input signal;
generating a correction sound by the electroacoustic output transducer of the hearing instrument on a basis of the correction signal to compensate for a structure-borne sound in the sound signal of the auditory canal; and
outputting the correction sound into the auditory canal of the test subject at least partially closed by the hearing instrument.

4. The method according to claim 3, which further comprises:
generating and outputting the correction sound as a broadband sound signal; and
ascertaining a residual noise signal not filtered by the active occlusion suppression as the second signal contribution on a basis of the first input signal.

5. The method according to claim 4, which further comprises:
recording an ambient sound of the hearing instrument by a second electroacoustic input transducer of the hearing instrument;
generating a second input signal from the ambient sound; and
carrying out active noise suppression in addition to the output of the test sound on a basis of the second input signal by the electroacoustic output transducer of the hearing instrument.

6. The method according to claim 5, which further comprises concentrating the active noise suppression on a frequency range around the at least one test frequency.

7. The method according to claim 6, wherein the active occlusion suppression by the correction signal takes place in a broadband manner.

8. A hearing system, comprising:
a hearing instrument, containing:
a first electroacoustic input transducer, being configured, when said hearing instrument is worn as intended by a test subject, to record a sound signal in an auditory canal of the test subject, and to generate a first input signal from the sound signal;
an electroacoustic output transducer configured to generate a test sound, and, when the hearing instrument is worn as intended, to output the test sound into the auditory canal of the test subject; and
a controller configured to register a reaction of the test subject to the test sound and to ascertain a hearing threshold of the test subject at at least one test frequency on a basis of the reaction of the test subject to the test sound and by means of the first input signal.

9. The hearing system according to claim 8, wherein:
said hearing instrument is configured as a hearing aid, and further contains a second electroacoustic input transducer, which is configured to record an ambient sound of said hearing aid, and to generate a second input signal from the ambient sound; and
said controller is configured to generate an output signal on a basis of the second input signal and supply the output signal to said electroacoustic output transducer for conversion into an output sound signal.

10. The hearing system according to claim 9, wherein said controller is configured to carry out active noise suppression on a basis of the second input signal by means of said electroacoustic output transducer in addition to the output of the test sound.
